(19) United States
(12) Patent Application Publication
(10) Pub. No.: US 2017/0007205 A1
(43) Pub. Date: Jan. 12, 2017

(54) ULTRASONIC DIAGNOSTIC APPARATUS AND PROGRAM

(71) Applicant: GE MEDICAL SYSTEMS GLOBAL TECHNOLOGY COMPANY, LLC, Waukesha, WI (US)

(72) Inventors: Shunichiro Tanigawa, Tokyo (JP); Atsuko Matsunaga, Tokyo (JP)

(21) Appl. No.: 15/113,134

(22) PCT Filed: Jan. 21, 2015
(86) PCT No.: PCT/US2015/012200

§ 371 Date: Jul. 21, 2016

(30) Foreign Application Priority Data
Jan. 21, 2014 (JP) 2014-008830

(51) Int. Cl. A61B 8/08; A61B 8/14; A61B 8/00; G01S 7/52

(52) U.S. Cl. CPC A61B 8/485; G01S 7/52042; G01S 7/52085; G01S 7/52022; A61B 8/14; A61B 8/461; A61B 8/5223

(57) ABSTRACT

To improve the frame rate of an ultrasonic diagnostic apparatus for displaying an elasticity image by detecting shear waves generated by push pulses. After first and second push pulses have been simultaneously transmitted to different positions, each of a first ultrasonic detecting pulse DP1 and a second ultrasonic detecting pulse DP2 for detecting each respective shear wave generated by each of the first and second push pulses is transmitted in a different acoustic line, and in a period after the first ultrasonic detecting pulse DP1 has been transmitted and before the first ultrasonic detecting pulse DP1 is transmitted again in the same acoustic line, the second ultrasonic detecting pulse DP2 is transmitted.

CROSS-REFERENCE TO RELATED APPLICATION SECTION

This application claims priority to Japan patent application number 2014-008830, filed on Jan. 21, 2014, the entirety of which is incorporated herein by reference.

### BACKGROUND

The present invention relates to an ultrasonic diagnostic apparatus and a program for measuring the elasticity of biological tissue by transmitting ultrasonic push pulses.

There have been known elasticity measurement techniques of measuring the elasticity of biological tissue by transmitting ultrasonic pulses (push pulses) having a high acoustic pressure from an ultrasonic probe to the biological tissue (for example, see Patent Document 1). More specifically, shear waves generated in biological tissue by push pulses are detected by ultrasonic detecting pulses, and the velocity of propagation of the shear waves and/or the elasticity value of the biological tissue are calculated to provide elasticity data. Then, an elasticity image having colors or the like according to the elasticity data is displayed.

To detect a shear wave, transmissions/receptions of a plurality of ultrasonic detecting pulses are conducted in the same acoustic line for a transmission of one push pulse. By each of the plurality of ultrasonic detecting pulses, the shear wave is detected at each portion in the acoustic line, thus providing elasticity data at several portions in the acoustic line.

In case that a two-dimensional elasticity image is to be displayed, transmissions/receptions of ultrasonic detecting pulses are conducted in a plurality of acoustic lines in a two-dimensional region for which the elasticity image is to be displayed. However, elasticity data in all acoustic lines in the two-dimensional region cannot always be acquired by a transmission of a single push pulse. In this case, a plurality of push pulses are transmitted, and after the transmission of each push pulse, transmissions/receptions of a plurality of ultrasonic detecting pulses are conducted in the same acoustic line.

Transmissions/receptions of the plurality of ultrasonic detecting pulses in the same acoustic line should be separated by a certain period of time. Thus, improvement of the frame rate is limited.

### BRIEF SUMMARY OF INVENTION

In an embodiment, an ultrasonic diagnostic apparatus includes a processor for executing a program conducting transmission control for, after transmitting an ultrasonic push pulse to biological tissue of a subject by an ultrasonic probe, transmitting a plurality of ultrasonic detecting pulses for detecting a shear wave generated in said biological tissue by said push pulse in the same acoustic line in said biological tissue by said ultrasonic probe, said processor executing a program conducting transmission control for, after simultaneously transmitting a plurality of said push pulses to different positions, transmitting each of said ultrasonic detecting pulses for detecting each respective shear wave generated by each of said push pulses in a different acoustic line, and in a transmission of each of said ultrasonic detecting pulses, transmitting, in a period after transmitting an ultrasonic detecting pulse for detecting a shear wave generated by one of said plurality of push pulses in a certain acoustic line and before transmitting said ultrasonic detecting pulse again in the same acoustic line, an ultrasonic detecting pulse for detecting a shear wave generated by a push pulse different from said push pulse in another acoustic line.

In an embodiment, a program causing a processor in an ultrasonic diagnostic apparatus to conduct a transmission control function of, after transmitting an ultrasonic push pulse to biological tissue of a subject by an ultrasonic probe, transmitting a plurality of ultrasonic detecting pulses for detecting a shear wave generated in said biological tissue by said push pulse in the same acoustic line in said biological tissue by said ultrasonic probe, wherein:

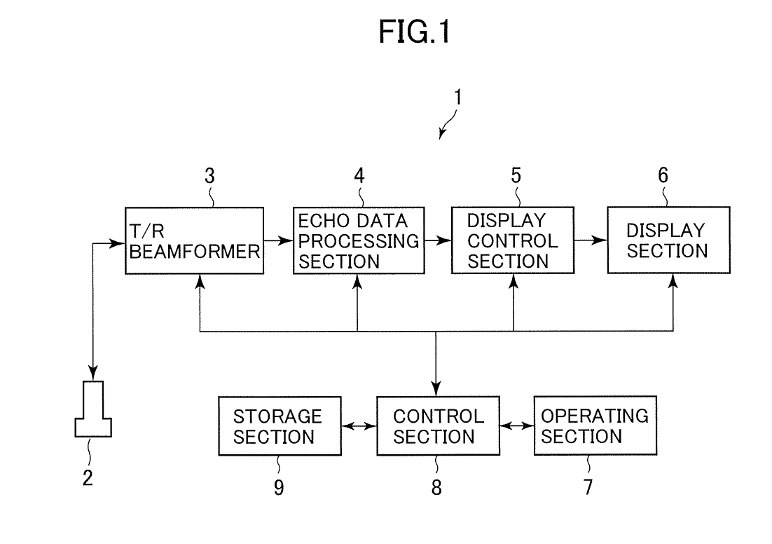

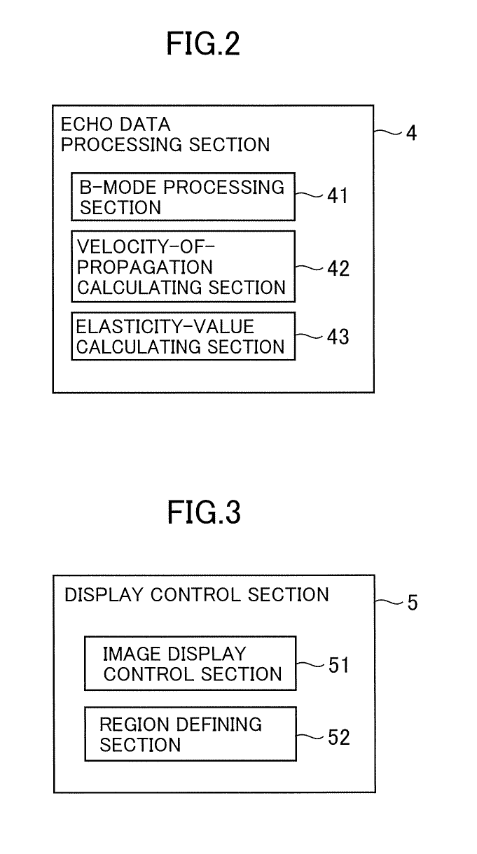

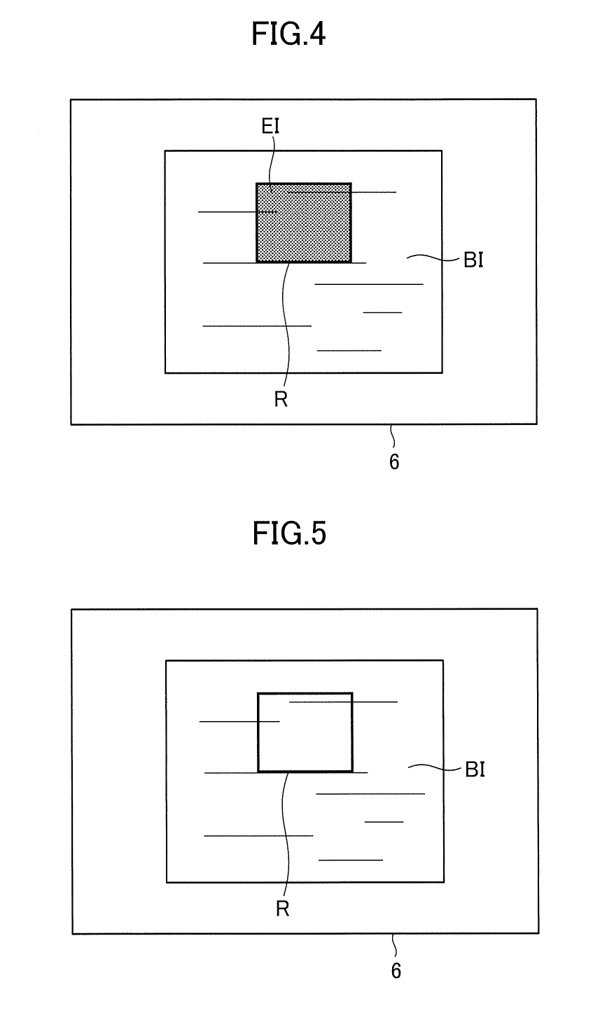

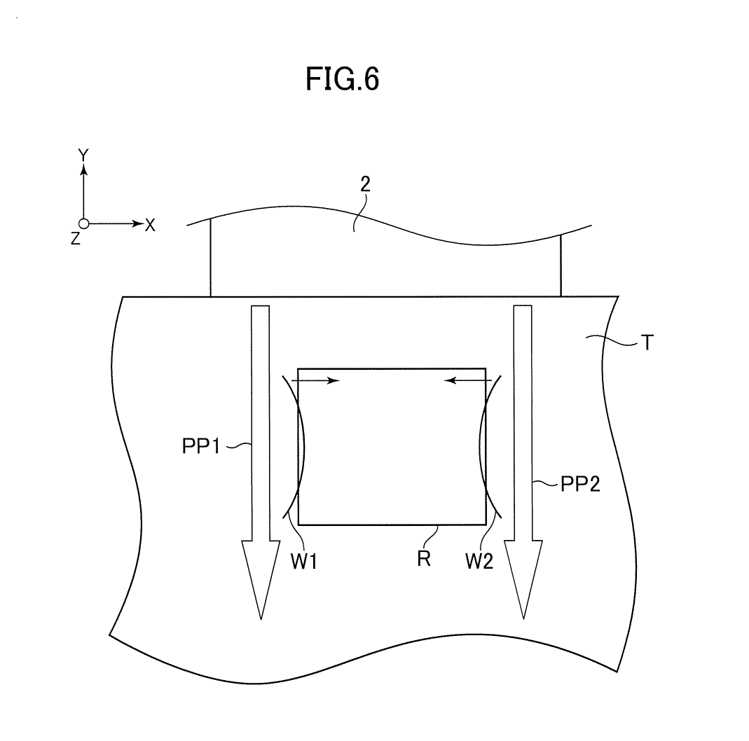

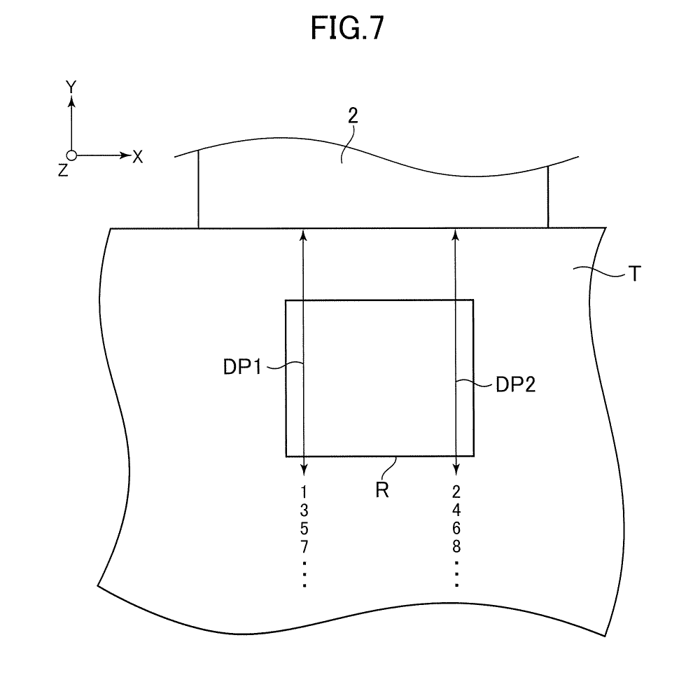

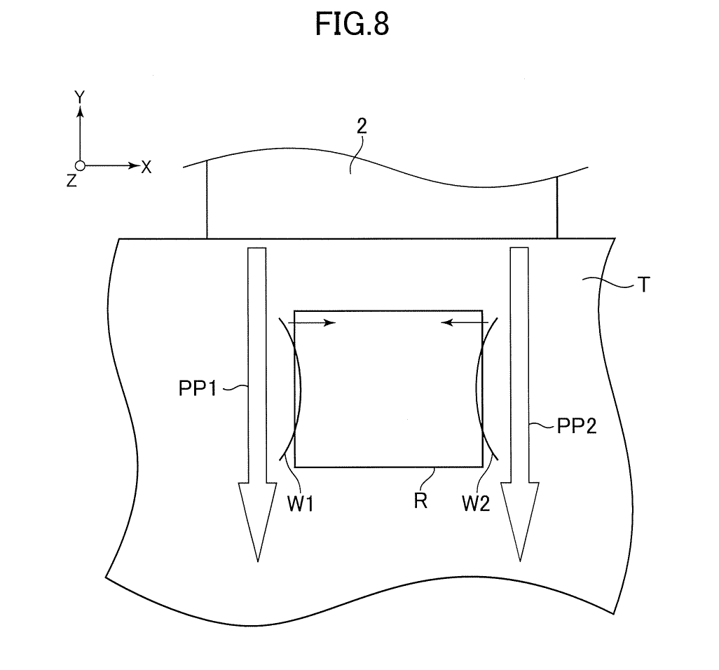

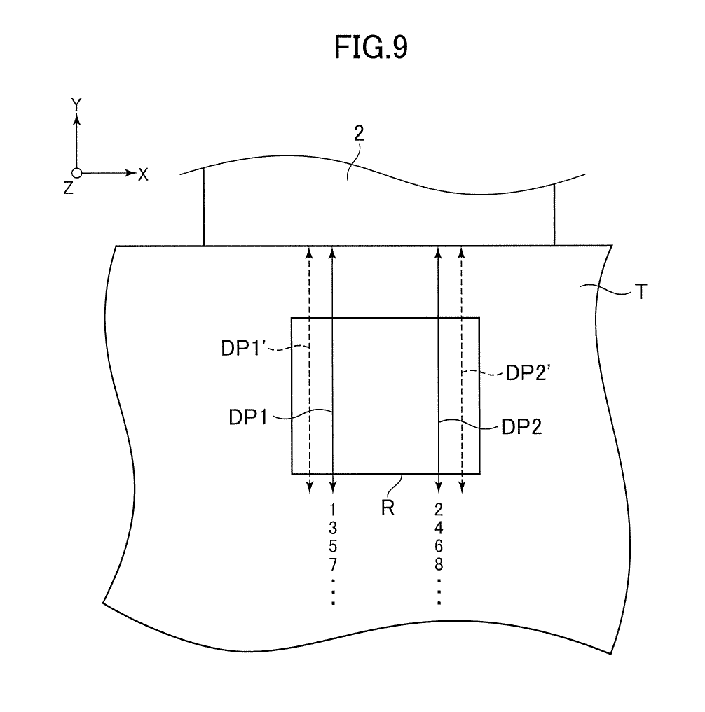

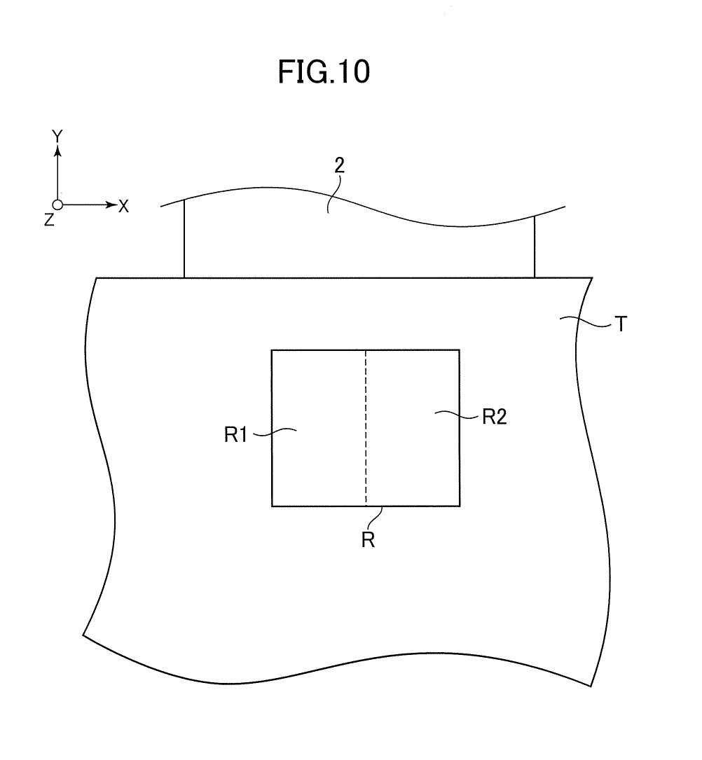

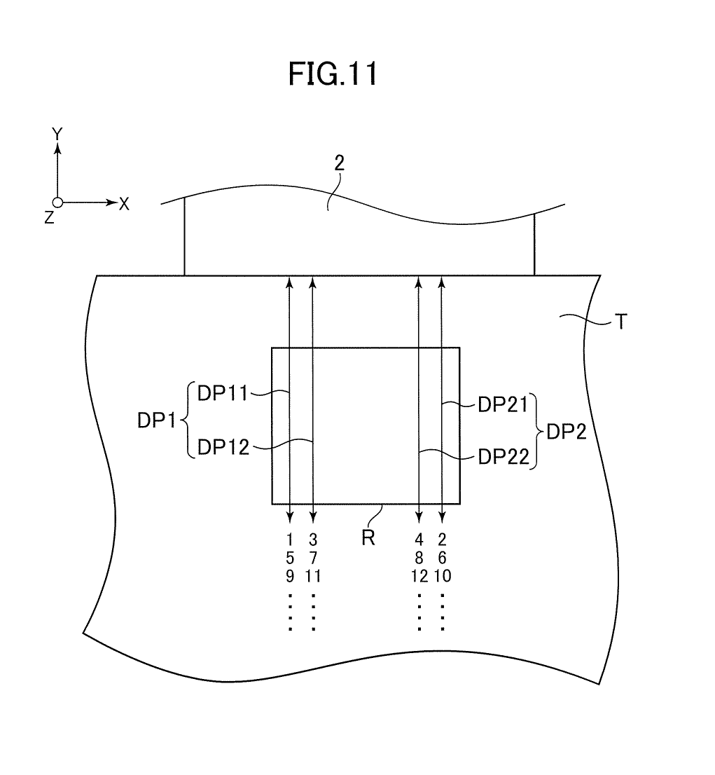

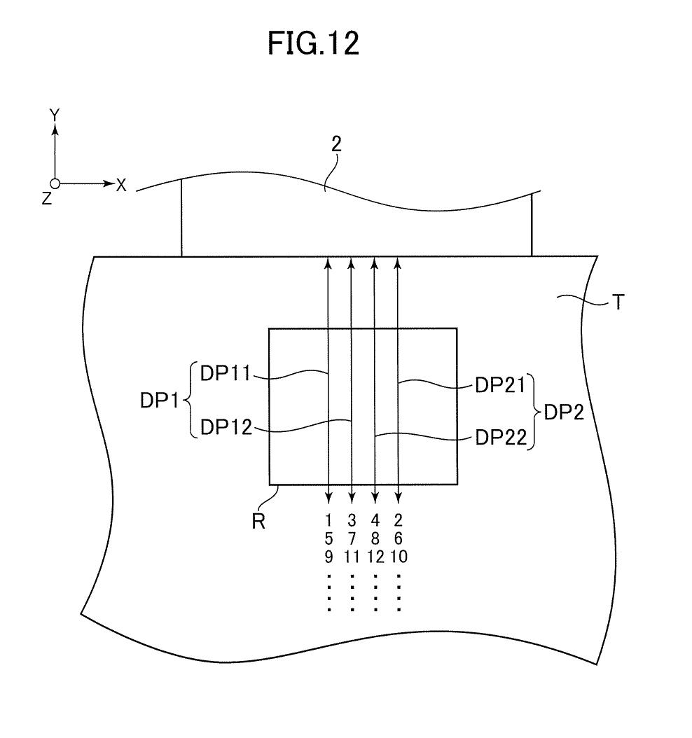

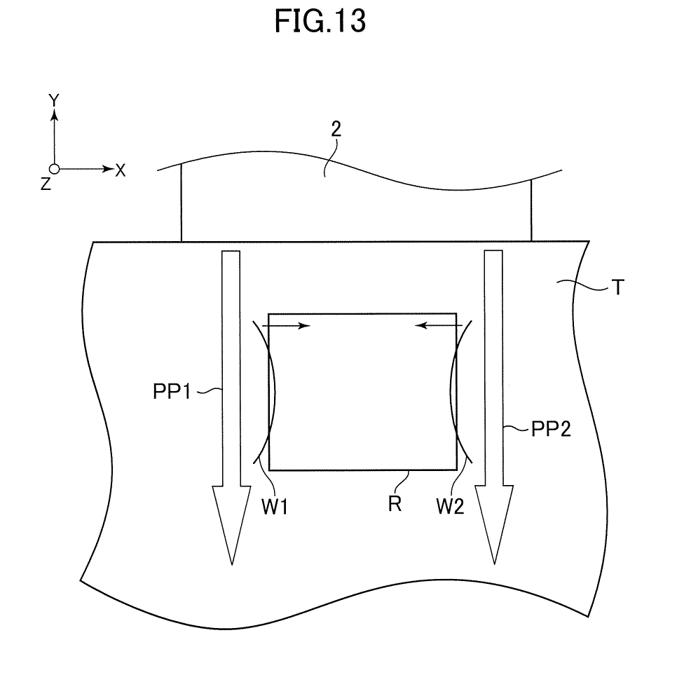

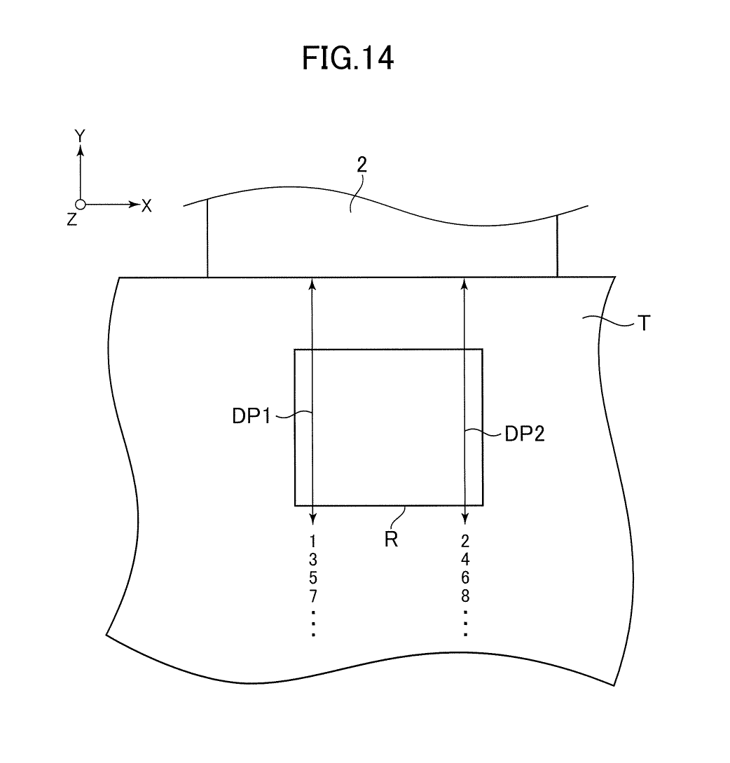

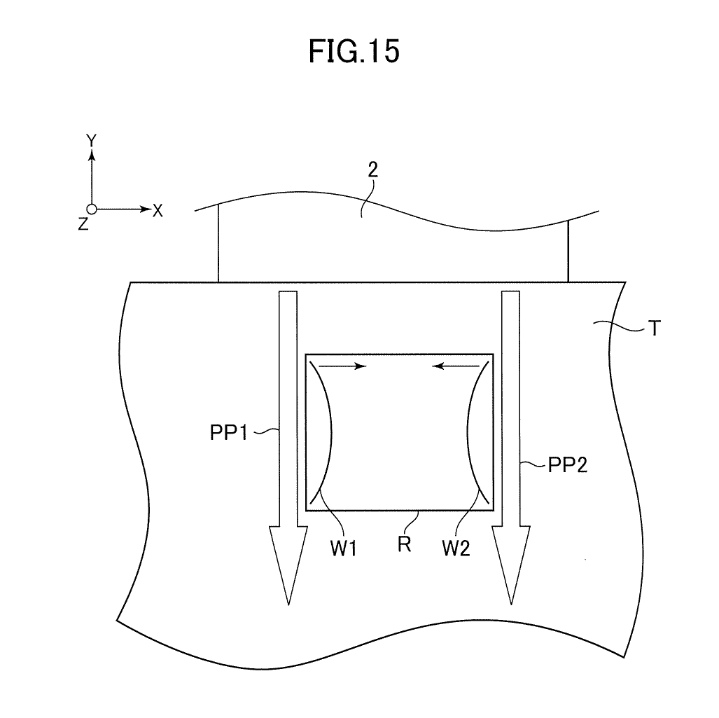

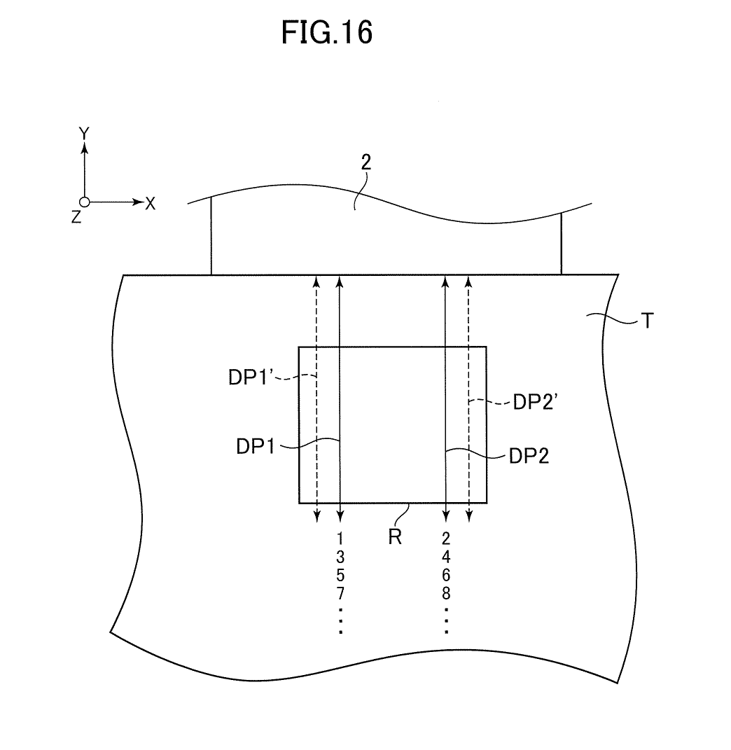

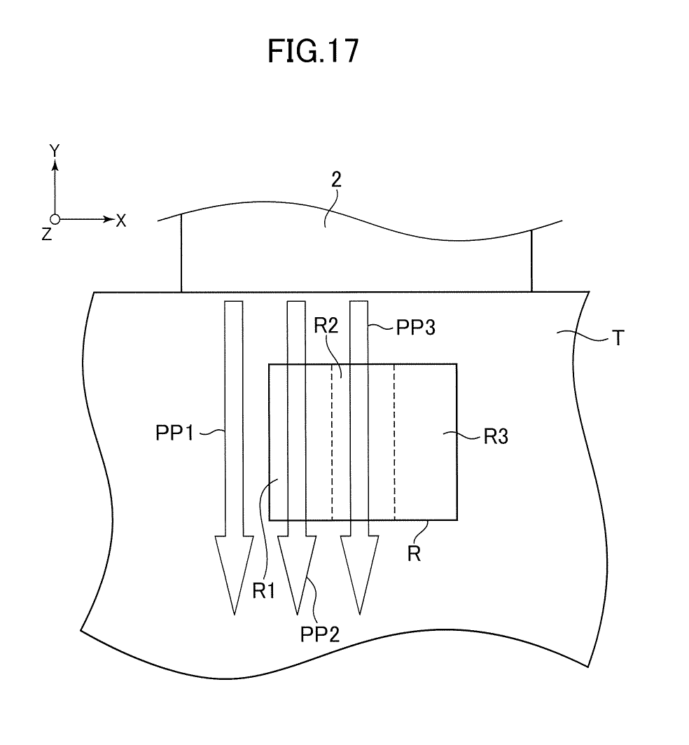

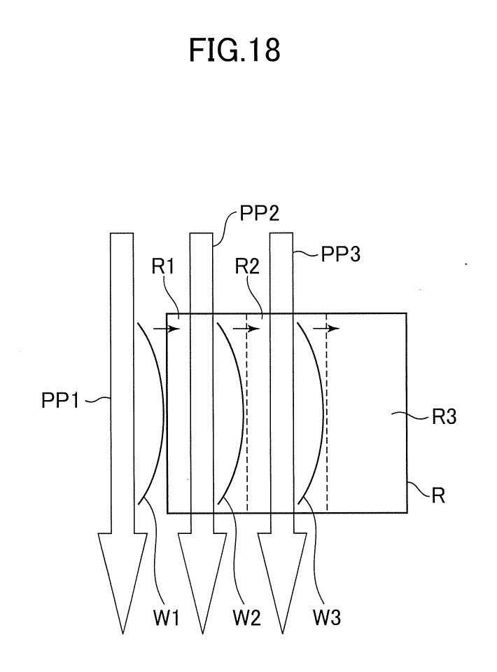

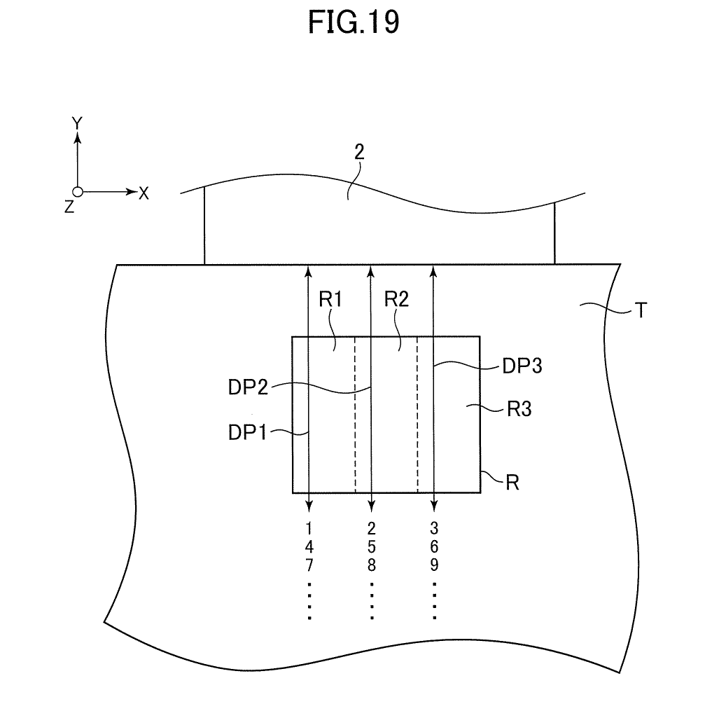

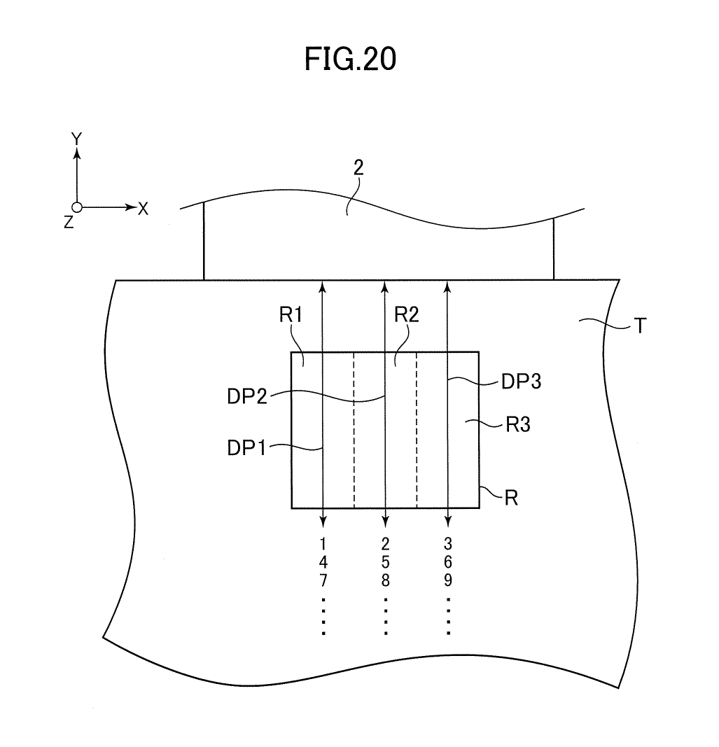

said transmission control function is a function of, after simultaneously transmitting a plurality of said push pulses to different positions, transmitting each of said ultrasonic detecting pulses for detecting each respective shear wave generated by each of said push pulses in a different acoustic line, and in a transmission of each of said ultrasonic detecting pulses, transmitting, in a period after transmitting an ultrasonic detecting pulse for detecting a shear wave generated by one of said plurality of push pulses in a certain acoustic line and before transmitting said ultrasonic detecting pulse again in the same acoustic line, an ultrasonic detecting pulse for detecting a shear wave generated by a push pulse different from said push pulse in another acoustic line.

[FIG. 1] A block diagram showing a schematic configuration of an ultrasonic diagnostic apparatus that is an exemplary embodiment of the present invention.

[FIG. 2] A block diagram showing a configuration of an echo data processing section.

[FIG. 3] A block diagram showing a configuration of a display control section.

[FIG. 4] A diagram showing a display section in which a B-mode image and an elasticity image are displayed.

[FIG. 5] A diagram showing the display section in which a region of interest is defined in the B-mode image.

[FIG. 6] A diagram for explaining a transmission of first and second push pulses, and shear waves generated by these push pulses.

[FIG. 7] A diagram for explaining transmissions/receptions of first and second ultrasonic detecting pulses.

[FIG. 8] A diagram for explaining a transmission of a second set of first and second push pulses.

[FIG. 9] A diagram for explaining transmissions/receptions of first and second ultrasonic detecting pulses corresponding to the second set of first and second push pulses.

[FIG. 10] A diagram for explaining a region to/from which the first and second ultrasonic detecting pulses are transmitted/received.

[FIG. 11] A diagram for explaining transmissions/receptions of first and second ultrasonic detecting pulses in a first variation of the first embodiment.

[FIG. 12] A diagram for explaining transmissions/receptions of the first and second ultrasonic detecting pulses corresponding to a second set of first and second push pulses.

[FIG. 13] A diagram for explaining a transmission of first and second push pulses in a second variation of the first embodiment.

[FIG. 14] A diagram for explaining transmissions/receptions of first and second ultrasonic detecting pulses.

[FIG. 15] A diagram for explaining a second set of first and second push pulses transmitted to positions different from those for the first set.

[FIG. 16] A diagram for explaining transmissions/receptions of first and second ultrasonic detecting pulses corresponding to the second set of first and second push pulses.

[FIG. 17] A diagram for explaining a transmission of first, second, and third push pulses in a second embodiment.

[FIG. 18] A diagram for explaining a first shear wave generated by the first push pulse, a second shear wave generated by the second push pulse, and a third shear wave generated by the third push pulse.

[FIG. 19] A diagram for explaining transmissions/receptions of first, second, and third ultrasonic detecting pulses.

[FIG. 20] A diagram for explaining transmissions/receptions of first, second, and third ultrasonic detecting pulses corresponding to a second set of first, second, and third push pulses.

### DETAILED DESCRIPTION OF THE INVENTION

Now embodiments of the present invention will be described hereinbelow.

To begin with, a first embodiment will be described. An ultrasonic diagnostic apparatus 1 shown in FIG. 1 comprises an ultrasonic probe 2, a T/R (transmission/reception) beamformer 3, an echo data processing section 4, a display control section 5, a display section 6, an operating section 7, a control section 8, and a storage section 9.

The ultrasonic probe 2 represents an exemplary embodiment of the ultrasonic probe in the present invention, which transmits ultrasound to biological tissue of a subject. By this ultrasonic probe 2, ultrasonic pulses (push pulses) for generating shear waves in the biological tissue are transmitted. Moreover, by the ultrasonic probe 2, ultrasonic detecting pulses for detecting the shear waves are transmitted and echo signals therefrom are received.

Further, by the ultrasonic probe 2, ultrasonic imaging pulses for producing a B-mode image are transmitted and echo signals therefrom are received.

The T/R beamformer 3 drives the ultrasonic probe 2 based on control signals from the control section 8 to transmit the several kinds of ultrasonic pulses with predetermined transmission parameters. The T/R beamformer 3 also applies signal processing such as phased addition processing to ultrasonic echo signals.

The echo data processing section 4 comprises a B-mode processing section 41, a velocity-of-propagation calculating section 42, and an elasticity-value calculating section 43, as shown in FIG. 2. The B-mode processing section 41 applies B-mode processing such as logarithmic compression processing and envelope detection processing to echo data output from the T/R beamformer 3, and creates B-mode data.

The velocity-of-propagation calculating section 42 calculates a velocity of propagation of the shear wave (velocity-of-propagation calculating function) based on echo data output from the T/R beamformer 3. The elasticity-value calculating section 43 calculates an elasticity value of the biological tissue to which push pulses are transmitted (elasticity-value calculating function) based on the velocity of propagation. Details thereof will be discussed later. The velocity of propagation and elasticity value represent an exemplary embodiment of the measured value with respect to the elasticity of biological tissue in the present invention.

It should be noted that only the velocity of propagation may be calculated without necessarily calculating the elasticity value. Data of the velocity of propagation or data of the elasticity value will be referred to herein as elasticity data.

The display control section 5 comprises an image display control section 51 and a region defining section 52, as shown in FIG. 3. The image display control section 51 scan-converts the B-mode data by a scan converter to create B-mode image data, based on which a B-mode image is displayed in the display section 6. The image display control section 51 also scan-converts the elasticity data by the scan converter to create elasticity image data, based on which an elasticity image is displayed in the display section 6.

As shown in FIG. 4, the elasticity image EI is a two-dimensional image displayed within a region of interest R defined in the B-mode image BI. The elasticity image EI is a color image having colors according to the velocity of propagation or elasticity value. The image display control section 51 combines the B-mode image data and elasticity image data together to create combined image data, based on which an image is displayed on the display section 6. Therefore, the elasticity image EI is a semi-transparent image through which the B-mode image BI in the background is allowed to pass.

The region of interest R is defined by the region defining section 52. More specifically, the region defining section 52 defines the region of interest R based on an input by an operator at the operating section 7. The region of interest R is a region for which shear waves are detected and in which transmissions/receptions of the ultrasonic detecting pulses are conducted.

The display section 6 is an LCD (Liquid Crystal Display), an organic EL (Electro-Luminescence) display, or the like. The operating section 7 is configured to comprise a pointing device for allowing the operator to input instructions and/or information, such as a keyboard, a trackball, and/or the like, although they are not specifically shown.

The control section 8 is a processor such as a CPU (Central Processing Unit). The control section 8 loads thereon a program stored in the storage section 9 and controls several sections in the ultrasonic diagnostic apparatus 1. For example, the control section 8 loads thereon a program stored in the storage section 9 and executes functions of the T/R beamformer 3, echo data processing section 4, and display control section 5 by the loaded program.

The control section 8 may execute all of the functions of the T/R beamformer 3, all of the functions of the echo data processing section 4, and all of the functions of the display control section 5 by the program, or execute only part of the functions by the program. In case that the control section 8 executes only part of the functions, the remaining functions may be executed by hardware such as circuitry.

It should be noted that the functions of the T/R beamformer 3, echo data processing section 4, and display control section 5 may be implemented by hardware such as circuitry.

The storage section 9 is an HDD (Hard Disk Drive), and/or a semiconductor memory such as a RAM (Random Access Memory) and/or a ROM (Read-Only Memory).

Next, an operation of the ultrasonic diagnostic apparatus 1 in the present embodiment will be described. First, an operator conducts ultrasound transmission/reception by the ultrasonic probe 2 on a subject to display a B-mode image BI based on echo signals, as shown in FIG. 5. The operator then makes an input at the operating section 7 to define a region of interest R in the B-mode image BI. Thus, the region of interest R is defined in the B-mode image BI. The region of interest R is defined as a region for which the operator desires to display an elasticity image.

Next, the operator makes an input at the operating section 7 to display an elasticity image. In response to this input, the control section 8 first drives the ultrasonic probe 2 to transmit a first push pulse PP1 and a second push pulse PP2, as shown in FIG. 6. The first push pulse PP1 and second push pulse PP2 are indicated by acoustic lines (arrows) in FIG. 6 (this similarly applies to the following drawings).

The first push pulse PP1 and second push pulse PP2 are simultaneously transmitted to different positions in the outside of the region of interest R. The first push pulse PP1 is transmitted to the vicinity of one edge of the region of interest R in an azimuthal direction (X direction) of the ultrasonic probe 2, and the second push pulse PP2 is transmitted to the vicinity of the other edge of the region of interest R in the azimuthal direction.

The first push pulse PP1 generates a first shear wave W1 in biological tissue T. The second push pulse PP2 generates a second shear wave W2 in the biological tissue T. The first shear wave W1 propagates within the biological tissue T in a direction away from the first push pulse PP1 (a direction of an arrow in FIG. 6). On the other hand, the second shear wave W2 propagates within the biological tissue T in a direction away from the second push pulse PP2 (a direction of an arrow in FIG. 6). Within the region of interest R, the first shear wave W1 propagates toward the second push pulse PP2, while the second shear wave W2 propagates toward the first push pulse PP1 in a direction opposite to that of the first shear wave W1.

After transmitting the first push pulse PP1 and second push pulse PP2, the control section 8 drives the ultrasonic probe 2 to transmit/receive a first ultrasonic detecting pulse DP1 and a second ultrasonic detecting pulse DP2 to/from the region of interest R, as shown in FIG. 7. The first ultrasonic detecting pulse DP1 and second ultrasonic detecting pulse DP2 are indicated by acoustic lines (arrows) in FIG. 7 (this similarly applies to the following drawings).

The first ultrasonic detecting pulse DP1 is an ultrasonic pulse for detecting the first shear wave W1 propagating within the region of interest R. The second ultrasonic detecting pulse DP2 is an ultrasonic pulse for detecting the second shear wave W2 propagating within the region of interest R.

The first ultrasonic detecting pulse DP1 and second ultrasonic detecting pulse DP2 are transmitted/received in respective ones of acoustic lines different from each other. The first ultrasonic detecting pulse DP1 is transmitted/received to/from the vicinity of one edge of the region of interest R in the azimuthal direction. On the other hand, the second ultrasonic detecting pulse DP2 is transmitted/received to/from the vicinity of the other edge of the region of interest R in the azimuthal direction.

The position of the first ultrasonic detecting pulse DP1 is on the right side to the first push pulse PP1, while the position of the second ultrasonic detecting pulse DP2 is on the left side to the second push pulse PP2. That is, the position of the first ultrasonic detecting pulse DP1 relative to the first push pulse PP1 and that of the second ultrasonic detecting pulse DP2 relative to the second push pulse PP2 are opposite to each other in the azimuthal direction. Therefore, the positions to/from which the first ultrasonic detecting pulse DP1 and second ultrasonic detecting pulse DP2 are transmitted/received are opposite to each other relative to their corresponding push pulses in the azimuthal direction. As used herein, a push pulse corresponding to the first ultrasonic detecting pulse DP1 is the first push pulse PP1, which is a source of generation of the first shear wave W1 detected by the first ultrasonic detecting pulse DP1. Likewise, a push pulse corresponding to the second ultrasonic detecting pulse DP2 is the second push pulse PP2, which is a source of generation of the second shear wave W2 detected by the second ultrasonic detecting pulse DP2.

The first ultrasonic detecting pulse DP1 is transmitted/received in an acoustic line lying at a predetermined distance D from the first push pulse PP1. The distance D is smaller than a distance from the first ultrasonic detecting pulse DP1 to the second push pulse PP2. The second ultrasonic detecting pulse DP2 is transmitted/received in an acoustic line lying at a predetermined distance D from the second push pulse PP2. The distance D is smaller than a distance from the second ultrasonic detecting pulse DP2 to the first push pulse PP1. The distance D from the first ultrasonic detecting pulse DP1 to the first push pulse PP1 and the distance D from the second ultrasonic detecting pulse DP2 to the second push pulse PP1 may be equal.

The control section 8 causes a plurality of the first ultrasonic detecting pulses DP1 and a plurality of second ultrasonic detecting pulses DP2 to be transmitted/received in respective ones of acoustic lines. In particular, in a period after transmitting/receiving the first ultrasonic detecting pulse DP1 for a start and before transmitting/receiving the first ultrasonic detecting pulse DP1 again in the same acoustic line, the control section 8 causes the second ultrasonic detecting pulse DP2 to be transmitted/received. Thus, the first ultrasonic detecting pulse DP1 and second ultrasonic detecting pulse DP2 are alternately transmitted/received. Numerals 1 through 8 in FIG. 7 designate the order of the first ultrasonic detecting pulse DP1 and second ultrasonic detecting pulse DP2 to be transmitted/received.

Now a reason why a plurality of the first ultrasonic detecting pulses DP1 and a plurality of the second ultrasonic detecting pulses DP2 are transmitted/received in respective ones of acoustic lines will be explained. The first shear wave W1 and second shear wave W2 cannot be detected at all points in respective acoustic lines by a transmission/reception of a single set of the first ultrasonic detecting pulse DP1 and second ultrasonic detecting pulse DP2. Accordingly, to detect the first shear wave W1 and second shear wave W2 at several points in respective acoustic lines, a plurality of the first ultrasonic detecting pulses DP1 and a plurality of the second ultrasonic detecting pulses DP2 are transmitted/received in respective ones of acoustic lines.

Once the control section 8 has conducted a specified number of times of transmissions/receptions of the first ultrasonic detecting pulses DP1 and second ultrasonic detecting pulses DP2, it causes the first push pulse PP1 and second push pulse PP2 to be transmitted again, as shown in FIG. 8. The positions to which the second set of the first push pulse PP1 and second push pulse PP2 are transmitted are the same as those for the first set.

Next, the control section 8 causes a first ultrasonic detecting pulse DP1 for detecting the first shear wave W1 (see FIG. 8) generated by the first push pulse PP1 in the second set and a second ultrasonic detecting pulse DP2 for detecting the second shear wave W2 (see FIG. 8) generated by the second push pulse PP2 in the second set to be transmitted/received, as shown in FIG. 9. The first ultrasonic detecting pulses DP1 and second ultrasonic detecting pulses DP2 corresponding to the first push pulse PP1 and second push pulse PP2 in the second set are transmitted/received in respective acoustic lines adjacent to those for the first set. In FIG. 9, dashed arrows indicate the first ultrasonic detecting pulse DP1′ and second ultrasonic detecting pulse DP2′ in the acoustic lines for the first set. The first ultrasonic detecting pulse DP1 in the acoustic line for the second set is closer to the second push pulse PP2 than the first ultrasonic detecting pulse DP1′ for the first set is. The second ultrasonic detecting pulse DP2 in the acoustic line for the second set is closer to the first push pulse PP1 than the second ultrasonic detecting pulse DP2′ for the first set is.

Transmissions/receptions of the first ultrasonic detecting pulses DP1 and second ultrasonic detecting pulses DP2 for the second set are also alternately conducted. Once a specified number of times of transmissions/receptions of the first ultrasonic detecting pulses DP1 and second ultrasonic detecting pulses DP2 have been conducted, the first push pulse PP1 and second push pulse PP2 are transmitted again. Thereafter, in a similar way, a transmission of the first push pulse PP1 and second push pulse PP2, and transmissions/receptions of their corresponding first ultrasonic detecting pulses DP1 and second ultrasonic detecting pulses DP2 are repeated. The first push pulse PP1 is transmitted to the same position every time, and the second push pulse PP2 is also transmitted to the same position every time. On the other hand, the first ultrasonic detecting pulses DP1 and second ultrasonic detecting pulses DP2 are transmitted/received in respective acoustic lines adjacent to those for the previous set within the region of interest R. Once transmissions/receptions of the first ultrasonic detecting pulses DP1 and second ultrasonic detecting pulses DP2 have been conducted in all acoustic lines within the region of interest R, transmissions/receptions of the first ultrasonic detecting pulses DP1 and second ultrasonic detecting pulses DP2 for producing an elasticity image in one frame are completed.

Now a region in which the first ultrasonic detecting pulses DP1 and second ultrasonic detecting pulses DP2 are transmitted/received will be explained. As shown in FIG. 10, one of two regions defined by equally dividing the region of interest R that lies near the first push pulse PP1 (not shown in FIG. 10) is designated as sub-region R1, and the other that lies near the second push pulse PP2 (not shown in FIG. 10) is designated as sub-region R2. The first ultrasonic detecting pulses DP1 are transmitted/received within the sub-region R1. The second ultrasonic detecting pulses DP2 are transmitted/received within the sub-region R2.

The velocity-of-propagation calculating section 42 calculates a velocity of propagation of the first shear wave W1 detected in echo signals for the first ultrasonic detecting pulses DP1 and a velocity of propagation of the second shear wave W2 detected in echo signals for the second ultrasonic detecting pulses DP2. The velocities of propagation are detected at portions corresponding to pixels in each of the acoustic lines.

The elasticity-value calculating section 43 calculates an elasticity value (Young's modulus (Pa: pascal)) at each portion based on the velocity of propagation. It should be noted that only the velocity of propagation may be calculated without calculating the elasticity value.

The image display control section 51 displays the elasticity image EI within the region of interest R in the display section 6 based on data of the velocity of propagation or data of the elasticity value (see FIG. 4).

According to the ultrasonic diagnostic apparatus 1 in the present embodiment, the frame rate can be improved. In particular, in a period after an ultrasonic detecting pulse for detecting a shear wave has been transmitted/received in one acoustic line and before the ultrasonic detecting pulse is transmitted/received again in the same acoustic line, a predetermined period of time for detecting the shear wave is placed. For example, in a period after the first ultrasonic detecting pulse DP1 has been transmitted/received and before the first ultrasonic detecting pulse DP1 is transmitted again, a predetermined period of time is placed. Accordingly, in the present embodiment, the period of time is efficiently used to transmit/receive the second ultrasonic detecting pulse DP2. Therefore, while elasticity data in the sub-region R1 is being acquired by a transmission/reception of the first ultrasonic detecting pulse DP1, elasticity data in the sub-region R2 can be acquired by a transmission/reception of the second ultrasonic detecting pulse DP2, and therefore, the frame rate can be doubled.

Next, variations of the first embodiment will be described. To begin with, a first variation will be described. In the first variation, for a set of a first push pulse PP1 and a second push pulse PP2, first ultrasonic detecting pulses DP1 and second ultrasonic detecting pulses DP2 are transmitted/received in respective pluralities of acoustic lines. For example, after the first push pulse PP1 and second push pulse PP2 have been simultaneously transmitted to different positions, the first ultrasonic detecting pulses DP1 and second ultrasonic detecting pulses DP2 are transmitted/received in respective two acoustic lines, as shown in FIG. 11.

Now transmissions/receptions of the first ultrasonic detecting pulses DP1 and second ultrasonic detecting pulses DP2 will be particularly described. As for the first ultrasonic detecting pulse DP1, first ultrasonic detecting pulses DP11, DP12 are transmitted/received in acoustic lines adjacent to each other. As for the second ultrasonic detecting pulse DP2, second ultrasonic detecting pulses DP21, DP22 are transmitted/received in acoustic lines adjacent to each other.

The control section 8 causes a plurality of the first ultrasonic detecting pulses DP11, DP12 and a plurality of the second ultrasonic detecting pulses DP21, DP22 to be transmitted/received in respective ones of acoustic lines. In particular, after transmitting/receiving the first ultrasonic detecting pulse DP11, second ultrasonic detecting pulse DP21, first ultrasonic detecting pulse DP12, and second ultrasonic detecting pulse DP22 in the order as described, the control section 8 causes the first ultrasonic detecting pulse DP11 to be transmitted/received again, and so on. Therefore, in the first variation, similarly to the embodiment described above, in a period after an ultrasonic detecting pulse for detecting a shear wave generated by one of a plurality of push pulses has been transmitted/received in a certain acoustic line and before the ultrasonic detecting pulse is transmitted/received again in the same acoustic line, an ultrasonic detecting pulse for detecting a shear wave generated by a push pulse different from the aforementioned push pulse is transmitted/received in another acoustic line. For example, in a period after the first ultrasonic detecting pulse DP11 has been transmitted/received and before the first ultrasonic detecting pulse DP11 is transmitted/received again in the same acoustic line, the second ultrasonic detecting pulse DP21 and second ultrasonic detecting pulse DP22 are transmitted/received.

Moreover, in the first variation, as described above, for example, in a period after the first ultrasonic detecting pulse DP11 has been transmitted/received and before the first ultrasonic detecting pulse DP11 is transmitted/received again in the same acoustic line, the first ultrasonic detecting pulse DP12 is transmitted/received. Therefore, in a period after an ultrasonic detecting pulse for detecting a shear wave generated by one of a plurality of push pulses has been transmitted/received in a certain acoustic line and before the ultrasonic detecting pulse is transmitted/received again in the same acoustic line, another ultrasonic detecting pulse for detecting a shear wave generated by that push pulse is transmitted/received in another acoustic line.

It should be noted that numerals 1 through 12 in FIG. 11 designate the order of the first ultrasonic detecting pulses DP11, DP12 and second ultrasonic detecting pulses DP21, DP22 to be transmitted/received.

Again in the present variation, once the control section 8 has conducted a specified number of times of transmissions/receptions of the first ultrasonic detecting pulses DP1 and second ultrasonic detecting pulses DP2, it causes the first push pulse PP1 and second push pulse PP2 to be transmitted again. Next, the control section 8 causes the first ultrasonic detecting pulses DP11, DP12 corresponding to the first push pulse PP1 and the second ultrasonic detecting pulses DP21, DP22 corresponding to the second push pulse PP2 to be transmitted/received again, as shown in FIG. 12. These first ultrasonic detecting pulses DP11, DP12 and second ultrasonic detecting pulses DP21, DP22 are transmitted/received a plurality of number of times in respective acoustic lines positioned differently from previous positions within the region of interest R. Once transmissions/receptions of the first ultrasonic detecting pulses DP11, DP12 and second ultrasonic detecting pulses DP21, DP22 have been conducted in all acoustic lines within the region of interest R, transmissions/receptions of the first ultrasonic detecting pulses DP1 and second ultrasonic detecting pulses DP2 for producing an elasticity image in one frame are completed.

Next, a second variation will be described. In the second variation, each time the first push pulse PP1 and second push pulse PP2 are transmitted, they are transmitted to positions different from previous positions. In particular, similarly to the first embodiment described above, for a start, the control section 8 causes the first push pulse PP1 and second push pulse PP2 to be simultaneously transmitted to the vicinity of one edge and the vicinity of the other edge in the region of interest R, as shown in FIG. 13.

Next, similarly to the embodiment described above, the control section 8 causes a plurality of first ultrasonic detecting pulses DP1 and a plurality of second ultrasonic detecting pulses DP2 to be alternately transmitted/received, as shown in FIG. 14. The first ultrasonic detecting pulses DP1 and second ultrasonic detecting pulses DP2 are transmitted/received in respective ones of acoustic lines. Then, after a specified number of times of transmissions/receptions of the first ultrasonic detecting pulses DP1 and second ultrasonic detecting pulses DP2 have been conducted, the control section 8 causes the first push pulse PP1 and second push pulse PP2 to be transmitted again, as shown in FIG. 15. The positions to which the second set of the first push pulse PP1 and second push pulse PP2 are transmitted are different from those for the first set. The positions of the first push pulse PP1 and second push pulse PP2 for the second set are such ones that their distances to corresponding first ultrasonic detecting pulse DP1 and second ultrasonic detecting pulse DP2 (a first ultrasonic detecting pulse DP1 and a second ultrasonic detecting pulse DP2 to be transmitted/received next, which will be described below; see FIG. 16) are the same as those for the first set.

Next, similarly to the embodiment described above, the control section 8 causes a plurality of first ultrasonic detecting pulses DP1 and a plurality of second ultrasonic detecting pulses DP2 to be alternately transmitted/received, as shown in FIG. 16. Thereafter, until the first ultrasonic detecting pulses DP1 and second ultrasonic detecting pulses DP2 are transmitted/received in all acoustic lines within the region of interest R, a transmission of the first push pulse PP1 and second push pulse PP2, and transmissions/receptions of their corresponding first ultrasonic detecting pulses DP1 and second ultrasonic detecting pulses DP2 are conducted. The first push pulse PP1 and second push pulse PP2 are transmitted such that their distances to corresponding first ultrasonic detecting pulse DP1 and second ultrasonic detecting pulse DP2 are equal.

Next, a second embodiment will be described. Particulars different from those in the first embodiment will be described hereinbelow.

In the present embodiment, the number and transmission positions of simultaneously transmitted push pulses are different from those in the first embodiment. In the present embodiment, the control section 8 causes a first push pulse PP1, a second push pulse PP2, and a third push pulse PP3 to be simultaneously transmitted to different positions, as shown in FIG. 17.

The positions of the first push pulse PP1, second push pulse PP2, and third push pulse PP3 will now be described. It is assumed that the region of interest R is divided into three sub-regions R1, R2, R3 for convenience of explanation. The sub-regions R1, R2, R3 are arranged side by side in this order from one edge of the region of interest R in an azimuthal direction. The first push pulse PP1 is transmitted in the outside of the sub-region R1 to the vicinity of one edge of the sub-region R1 in the azimuthal direction (on the left side to the sub-region R1). The second push pulse PP2 is transmitted in the outside of the sub-region R2 to the vicinity of one edge of the sub-region R2 in the azimuthal direction (on the left side to the sub-region R2). The third push pulse PP3 is transmitted in the outside of the sub-region R3 to the vicinity of one edge of the sub-region R3 in the azimuthal direction (on the left side to the sub-region R3).

As shown in FIG. 18, the first push pulse PP1 generates a first shear wave W1 propagating in a direction away from the first push pulse PP1 (a direction of an arrow in FIG. 18). The second push pulse PP2 generates a second shear wave W2 propagating in a direction away from the second push pulse PP2 (a direction of an arrow in FIG. 18). The third push pulse PP3 generates a third shear wave W3 propagating in a direction away from the third push pulse PP3 (a direction of an arrow in FIG. 18).

The first shear wave W1 is detected by first ultrasonic detecting pulses DP1, which will be discussed later, within the sub-region R1. The second shear wave W2 is detected by second ultrasonic detecting pulses DP2, which will be discussed later, within the sub-region R2. The third shear wave W3 is detected by third ultrasonic detecting pulses DP3, which will be discussed later, within the sub-region R3.

After transmitting the first push pulse PP1, second push pulse PP2, and third push pulse PP3, the control section 8 drives the ultrasonic probe 2 to transmit/receive a first ultrasonic detecting pulse DP1 for detecting the first shear wave W1 (not shown in FIG. 19) propagating within the sub-region R1, a second ultrasonic detecting pulse DP2 for detecting the second shear wave W2 (not shown in FIG. 19) propagating within the sub-region R2, and a third ultrasonic detecting pulse DP3 for detecting the third shear wave W3 (not shown in FIG. 19) propagating within the sub-region R3, as shown in FIG. 19.

The first ultrasonic detecting pulse DP1, second ultrasonic detecting pulse DP2, and third ultrasonic detecting pulse DP3 are transmitted/received in respective ones of acoustic lines different from one another. The first ultrasonic detecting pulse DP1 is transmitted/received within the sub-region R1 to/from the vicinity of one edge (near the first push pulse PP1) of the sub-region R1 in the azimuthal direction. The second ultrasonic detecting pulse DP2 is transmitted/received within the sub-region R2 to/from the vicinity of one edge (near the second push pulse PP2) of the sub-region R2 in the azimuthal direction. The third ultrasonic detecting pulse DP3 is transmitted/received within the sub-region R3 to/from the vicinity of one edge (near the third push pulse PP3) of the sub-region R3 in the azimuthal direction.

The position of the first ultrasonic detecting pulse DP1 is on the right side to the first push pulse PP1. The position of the second ultrasonic detecting pulse DP2 is on the right side to the second push pulse PP2. The position of the third ultrasonic detecting pulse DP3 is on the right side to the third push pulse PP3. That is, the position of the first ultrasonic detecting pulse DP1 relative to the first push pulse PP1, the position of the second ultrasonic detecting pulse DP2 relative to the second push pulse PP2, and the position of the third ultrasonic detecting pulse DP3 relative to the third push pulse PP3 are on the same side as one another in the azimuthal direction. Therefore, in the present embodiment, the positions to/from which the first ultrasonic detecting pulse DP1, second ultrasonic detecting pulse DP2, and third ultrasonic detecting pulse DP3 are transmitted/received are on the same side as one another in the azimuthal direction relative to their corresponding push pulses.

The control section 8 causes a plurality of the first ultrasonic detecting pulses DP1, a plurality of the second ultrasonic detecting pulses DP2, and a plurality of the third ultrasonic detecting pulse DP3 to be transmitted/received in respective ones of acoustic lines. In particular, in a period after transmitting/receiving the first ultrasonic detecting pulse DP1 for a start and before transmitting/receiving the first ultrasonic detecting pulse DP1 again in the same acoustic line, the control section 8 causes the second ultrasonic detecting pulse DP2 and third ultrasonic detecting pulse DP3 to be transmitted/received in this order. Therefore, transmissions/receptions are conducted in the order of the first ultrasonic detecting pulse DP1, second ultrasonic detecting pulse DP2, and third ultrasonic detecting pulse DP3. Numerals 1 through 9 in FIG. 19 designate the order of the first ultrasonic detecting pulse DP1, second ultrasonic detecting pulse DP2, and third ultrasonic detecting pulse DP3 to be transmitted/received.

Once the control section 8 has conducted a specified number of transmissions/receptions of the first ultrasonic detecting pulses DP1, second ultrasonic detecting pulses DP2, and third ultrasonic detecting pulses DP3, it causes the first push pulse PP1, second push pulse PP2, and third push pulse PP3 to be transmitted again. The positions to which the second set of the first push pulse PP1, second push pulse PP2, and third push pulse PP3 are transmitted may be the same as or different from those for the first set. In case that the first push pulse PP1, second push pulse PP2, and third push pulse PP3 are transmitted to different positions between the first and second sets, the positions for the first push pulse PP1, second push pulse PP2, and third push pulse PP3 are such ones that their distances to corresponding first ultrasonic detecting pulse DP1, second ultrasonic detecting pulse DP2, and third ultrasonic detecting pulse DP3 (a first ultrasonic detecting pulse DP1, a second ultrasonic detecting pulse DP2, and a third ultrasonic detecting pulse DP3 to be transmitted/received next, which will be described below; see FIG. 20) are the same as those for the first set.

Next, the control section 8 causes a first ultrasonic detecting pulse DP1 for detecting the first shear wave W1 generated by the first push pulse PP1 in the second set, a second ultrasonic detecting pulse DP2 for detecting the second shear wave W2 generated by the second push pulse PP2 in the second set, and a third ultrasonic detecting pulse DP3 for detecting the third shear wave W3 generated by the third push pulse PP3 in the second set to be transmitted/received, as shown in FIG. 20. The first ultrasonic detecting pulses DP1, second ultrasonic detecting pulses DP2, and third ultrasonic detecting pulses DP3 corresponding to the second set of the first push pulse PP1, second push pulse PP2, and third push pulse PP3 are transmitted/received in respective acoustic lines adjacent to those for the first set (not shown in FIG. 20), as in the first embodiment.

Transmissions/receptions of the first ultrasonic detecting pulses DP1, second ultrasonic detecting pulses DP2, and third ultrasonic detecting pulses DP3 for the second set are conducted a specified number of times in this order, as in the first set. Thereafter, in a similar way, a transmission of the first push pulse PP1, second push pulse PP2, and third push pulse PP3, and transmissions/receptions of their corresponding first ultrasonic detecting pulses DP1, second ultrasonic detecting pulses DP2, and third ultrasonic detecting pulses DP3 are repeated. Once transmissions/receptions of the first ultrasonic detecting pulses DP1, second ultrasonic detecting pulses DP2, and third ultrasonic detecting pulses DP3 have been conducted in all acoustic lines within the region of interest R, transmissions/receptions of the first ultrasonic detecting pulses DP1, second ultrasonic detecting pulses DP2, and third ultrasonic detecting pulses DP3 for producing an elasticity image in one frame are completed.

According to the ultrasonic diagnostic apparatus 1 in the present embodiment, again, in a period after the first ultrasonic detecting pulse DP1 has been transmitted/received and before the first ultrasonic detecting pulse DP1 is transmitted again in the same acoustic line, the second ultrasonic detecting pulse DP2 and third ultrasonic detecting pulse DP3 are transmitted/received. Thus, the frame rate can be improved threefold.

In the present embodiment, similarly to the first variation of the first embodiment, for one push pulse, corresponding ultrasonic detecting pulses may be transmitted/received in a plurality of acoustic lines.

While the present invention has been described with reference to the embodiments, it will be easily recognized that the present invention may be practiced with several modifications without departing from the spirit and scope thereof. For example, in the second embodiment, the number of simultaneously transmitted push pulses is not limited to three and any plural number of push pulses may be applied.

1. An ultrasonic diagnostic apparatus comprising:
a processor for executing a program conducting transmission control for, after transmitting an ultrasonic push pulse to biological tissue of a subject by an ultrasonic probe, transmitting a plurality of ultrasonic detecting pulses for detecting a shear wave generated in said biological tissue by said push pulse in the same acoustic line in said biological tissue by said ultrasonic probe;
said processor executing a program conducting transmission control for, after simultaneously transmitting a plurality of said push pulses to different positions, transmitting each of said ultrasonic detecting pulses for detecting each respective shear wave generated by each of said push pulses in a different acoustic line;
and in a transmission of each of said ultrasonic detecting pulses, transmitting, in a period after transmitting an ultrasonic detecting pulse for detecting a shear wave generated by one of said plurality of push pulses in a certain acoustic line and before transmitting said ultrasonic detecting pulse again in the same acoustic line;
an ultrasonic detecting pulse for detecting a shear wave generated by a push pulse different from said push pulse in another acoustic line.

2. The ultrasonic diagnostic apparatus as recited in claim 1, further comprising: a display section in which an elasticity image having a mode of display according to the elasticity of biological tissue based on echo signals for said ultrasonic detecting pulses is displayed in a two-dimensional region.

3. The ultrasonic diagnostic apparatus as recited in claim 2, wherein said ultrasonic detecting pulses are transmitted to said two-dimensional region, and an ultrasonic detecting pulse for detecting a shear wave generate by one of said plurality of push pulses is transmitted in an acoustic line lying at a predetermined distance from said push pulse, wherein said predetermined distance is smaller than a distance from said ultrasonic detecting pulse to a push pulse different from said push pulse.

4. The ultrasonic diagnostic apparatus as recited in claim 1, wherein said processor executes a program for transmitting said ultrasonic detecting pulses in one acoustic line for one said push pulse.

5. The ultrasonic diagnostic apparatus of claim 1, wherein said processor executes a program for transmitting said ultrasonic detecting pulses in a plurality of acoustic lines for one said push pulse.

6. The ultrasonic diagnostic apparatus as recited in claim 5, wherein said processor executes a program conducting transmission control for, in a period after transmitting an ultrasonic detecting pulse for detecting a shear wave generated by one of said plurality of push pulses in a certain acoustic line and before transmitting said ultrasonic detecting pulse again in the same acoustic line, transmitting another ultrasonic detecting pulse for detecting a shear wave generated by said push pulse in another acoustic line.

7. The ultrasonic diagnostic apparatus of claim 2, wherein said two-dimensional region is divided into a plurality of sub-regions, and each of said ultrasonic detecting pulses for detecting a shear wave generated by each of said plurality of push pulses is transmitted in each respective sub-region.

8. The ultrasonic diagnostic apparatus of claim 1, wherein a position to which each of said ultrasonic detecting pulses for detecting a shear wave generated by each of said plurality of push pulses simultaneously transmitted to different positions is transmitted is on a side opposite to each other with respect to a corresponding push pulse in an azimuthal direction of said ultrasonic probe.

9. The ultrasonic diagnostic apparatus of claim 1, wherein a position to which each of said ultrasonic detecting pulses for detecting a shear wave generated by each of said plurality of push pulses simultaneously transmitted to different positions is transmitted is on the same side as each other with respect to a corresponding push pulse in an azimuthal direction of said ultrasonic probe.

10. The ultrasonic diagnostic apparatus of claim 2, wherein said processor executes a program of a measurement-value calculating function for calculating a measured value with respect to the elasticity of said biological tissue based on echo signals for said ultrasonic detecting pulses.

11. The ultrasonic diagnostic apparatus as recited in claim 10, wherein said measurement-value calculating function is a velocity-of-propagation calculating function of calculating a velocity of propagation of said shear wave.

12. The ultrasonic diagnostic apparatus as recited in claim 10, wherein said measurement-value calculating function is an elasticity-value calculating function of calculating an elasticity value of biological tissue based on the velocity of propagation of said shear waves.

13. The ultrasonic diagnostic apparatus of claim 10, wherein said elasticity image has a mode of display according to said measured value.

14. A program causing a processor in an ultrasonic diagnostic apparatus to conduct a transmission control function of, after transmitting an ultrasonic push pulse to biological tissue of a subject by an ultrasonic probe, transmitting a plurality of ultrasonic detecting pulses for detecting a shear wave generated in said biological tissue by said push pulse in the same acoustic line in said biological tissue by said ultrasonic probe, wherein:
said transmission control function is a function of, after simultaneously transmitting a plurality of said push pulses to different positions, transmitting each of said ultrasonic detecting pulses for detecting each respective shear wave generated by each of said push pulses in a different acoustic line;
and in a transmission of each of said ultrasonic detecting pulses, transmitting, in a period after transmitting an ultrasonic detecting pulse for detecting a shear wave generated by one of said plurality of push pulses in a certain acoustic line and before transmitting said ultrasonic detecting pulse again in the same acoustic line, an ultrasonic detecting pulse for detecting a shear wave generated by a push pulse different from said push pulse in another acoustic line.
